(19) United States
(12) Patent Application Publication
(10) Pub. No.: US 2014/0105838 A1
(43) Pub. Date: Apr. 17, 2014

(54) BLOCK POLYMER INCLUDING ISOBUTYL ACRYLATE AND ACRYLIC ACID, COSMETIC COMPOSITION AND TREATMENT METHOD

(71) Applicants: Celine Farcet, Paris (FR); Bertrand Lion, Paris (FR)

(72) Inventors: Celine Farcet, Paris (FR); Bertrand Lion, Paris (FR)

(73) Assignee: L'OREAL, Paris (FR)

(21) Appl. No.: 14/116,015

(22) PCT Filed: May 11, 2012
(86) PCT No.: PCT/FR2012/051055

§ 371 Date: Dec. 6, 2013

Related U.S. Application Data
(60) Provisional application No. 61/487,751, filed on May 19, 2011.

(30) Foreign Application Priority Data
May 13, 2011 (FR) 1154161

(51) Int. Cl. C08F 20/10; A61Q 1/02; A61Q 19/00; A61Q 5/00; A61Q 1/04; A61Q 1/10; A61Q 3/02; A61Q 19/04; A61K 8/90; A61Q 17/04

(52) U.S. Cl. CPC C08F 20/10; A61K 8/90; A61Q 1/02; A61Q 19/007; A61Q 17/04; A61Q 19/00; A61Q 1/04; A61Q 1/10; A61Q 3/02; A61Q 19/04; A61Q 5/00; A61Q 19/001
USPC 424/59; 525/289; 525/288; 424/78.02; 424/78.03; 424/63; 424/64; 424/61; 424/70.1

(57) ABSTRACT

The present invention relates to a diblock or triblock polymer of AB, ABA or BAB structure, comprising at least one block A which comprises isobutyl acrylate and acrylic acid. The invention also relates to a cosmetic composition comprising, in a cosmetically acceptable medium, at least one such polymer, and also to a treatment process using it.

The present invention relates to novel block polymers, and also to the cosmetic compositions comprising them, and to a cosmetic treatment process using them.

Block polymers which can be carried in a non-aqueous medium, and which are in particular soluble or dispersed in said medium, have film-forming properties which are advantageous but can be penalized in applications in the make-up field, in particular by virtue of their sensitivities to fatty substances, such as food oils or sebum.

The objective of the present invention is to provide block polymers with low sensitivity to fatty substances, in particular to food oils and to sebum.

It has in fact been noted that the presence of certain monomers within the same block, linked to a particular configuration of the polymer (succession of the blocks), can make it possible to achieve this objective.

The subject of the present invention is therefore a diblock or triblock polymer of AB, ABA or BAB structure, comprising at least one block A which comprises isobutyl acrylate and acrylic acid.

The invention also relates to a cosmetic composition comprising, in a cosmetically acceptable medium, at least one such polymer.

The polymers according to the invention are preferably film-forming polymers which have low sensitivity to food oils or are insensitive thereto, and therefore capable of being advantageously used in make-up compositions.

The term “film-forming polymer” is intended to mean a polymer that is capable of forming, by itself or in the presence of an auxiliary film-forming agent, a continuous deposit on a support, in particular on keratin materials.

The polymers according to the invention have, moreover, good resistance to olive oil (food proof), improved compared with the known block polymers.

The block polymer according to the invention has a structure chosen from the following structures: AB, BA and BAB.

It can therefore comprise two distinct blocks and therefore be a diblock polymer which can be symbolized AB comprising a block A and a block B.

It can also comprise three distinct blocks and therefore be a triblock polymer which can be symbolized either ABA (or ABA′) when it comprises a central block B and two external blocks A (or A and A′), which may or may not be identical, but are generally identical; or BAB (or BAB′) when it comprises a central block A and two external blocks B (or B and B′), which may or may not be identical, but are generally identical.

Preferably, it is an ethylenic polymer, i.e. a polymer obtained by polymerization of monomers comprising an ethylenic unsaturation, preferably just one ethylenic unsaturation per monomer.

Preferably, the block polymer is linear (in particular without intentional addition of crosslinker and/or of difunctional monomer).

The block polymer according to the invention is characterized in particular in that it comprises a block (referred to hereinafter as block A) which comprises both isobutyl acrylate units and acrylic acid units.

Preferably, the isobutyl acrylate represents 75% to 99.5% by weight, in particular 78% to 95% by weight, of the total weight of the isobutyl acrylate+acrylic acid mixture.

Preferably, the acrylic acid represents 0.5% to 25% by weight, in particular 5% to 22% by weight, of the total weight of the isobutyl acrylate+acrylic acid mixture.

Preferably, the isobutyl acrylate+acrylic acid mixture represents 50% to 100% by weight, in particular 60% to 90% by weight, or even 70% to 80% by weight, of the total weight of said block.

This block A can therefore comprise additional monomers, which may be present in a proportion of from 0 to 50% by weight, in particular 10% to 40% by weight, or even 20% to 30% by weight, in said block.

These additional monomers are preferably chosen from, alone or as a mixture, the following monomers and also the salts thereof:

(i) (meth)acrylates of formula CH₂═CHCOOR or CH₂═C(CH₃)COOR in which R represents:

a linear or branched alkyl group comprising 1 to 30 carbon atoms, optionally inserted into which is (are) one or more heteroatoms chosen from O, N, S and P, and/or it being possible for said alkyl group to be optionally substituted with one or more substituents chosen from —OH, halogen atoms (Cl, Br, I and F), —NR4R5 groups, where R4 and R5, which may be identical or different, represent hydrogen or a linear or branched C₁-C₆ alkyl group or a phenyl group; and/or polyoxyalkylene, in particular polyoxyethylene and/or polyoxypropylene, groups, said polyoxyalkylene group consisting of 5 to 30 repeating oxyalkylene units;

a C₃-C₁₂ cycloalkyl group, it being possible for said cycloalkyl group to comprise in its chain one or more heteroatoms chosen from O, N, S and/or P, and/or to be optionally substituted with one or more substituents chosen from —OH and halogen atoms (Cl, Br, I and F);

a C₄-C₂₀ aryl or C₅-C₃₀ aralkyl group (C₁-C₈ alkyl group);

with the exclusion of isobutyl acrylate;

(ii) (meth)acrylamides of formula CH₂═CHCONR₁R₂ or CH₂═C(CH₃)CONR₁R₂ in which R1 and R2, which may be identical or different, represent H or:

a linear or branched alkyl group comprising from 1 to 18 carbon atoms, optionally inserted into which is (are) one or more heteroatoms chosen from O, N, S and P; it being possible for said alkyl group to be substituted with one or more substituents chosen from OH and halogen atoms (Cl, Br, I and F), and/or to be interrupted with an SiR4R5 group, where R4 and R5, which may be identical or different, represent a C₁-C₆ alkyl group or a phenyl group;

and in particular R1 and/or R2 can be a methyl, ethyl, propyl, isopropyl, n-butyl, isobutyl, tert-butyl, hexyl, isohexyl, cyclohexyl, ethylhexyl, octyl, isooctyl, decyl, isodecyl, cyclodecyl, dodecyl, cyclododecyl, isononyl, lauryl, t-butylcyclohexyl, stearyl or ethyl-2-perfluorohexyl group; or a C₁-C₄ hydroxyalkyl group such as 2-hydroxyethyl, 2-hydroxybutyl or 2-hydroxypropyl; or a (C₁-C₄)alkoxy(C₁-C₄)alkyl group such as methoxyethyl, ethoxyethyl or methoxypropyl,

a C₃-C₁₂ cycloalkyl group, such as isobornyl, or a heterocycloalkyl group (C₁-C₄ alkyl), such as furfurylmethyl or tetrahydrofurfurylmethyl,

a C₄-C₂₀ aryl group such as the phenyl group,

a C₅-C₃₀ aralkyl group (C₁-C₈ alkyl group) such as 2-phenylethyl, t-butylbenzyl or benzyl;

(iii) monomers with ethylenic unsaturation(s) comprising at least one carboxylic, phosphoric or sulfonic acid function, or anhydride function, for instance methacrylic acid, crotonic acid, maleic anhydride, itaconic acid, fumaric acid, maleic acid, styrenesulfonic acid, vinylbenzoic acid, vinylphosphoric acid or acrylamidopropanesulfonic acid, and salts thereof, with the exclusion of acrylic acid;

(iv) vinyl ethers of formula R₆O—CH═CH₂ or vinyl esters of formula: R₆—COO—CH═CH₂ in which R₆ represents a linear or branched alkyl group comprising from 1 to 22 atoms, or a cyclic alkyl group comprising from 3 to 6 carbon atoms and/or an aromatic group, for example of benzene, anthracene or naphthalene type;

(v) vinyl compounds of formulae CH₂═CH—R9, CH₂═CH—CH₂—R9 or CH₂═C(CH₃)—CH₂—R9 in which R9 is:

a hydroxyl, halogen (Cl or F), NH₂ or acetamide (NHCOCH₃) group;

an OCOR11 group where R11 represents a linear or branched C₂-C₁₂ alkyl group;

an OR10 group where R10 represents a phenyl group or a C₁-C₁₂ alkyl group;

a linear or branched C₁-C₁₈ alkyl group, optionally inserted into which is (are) one or more heteroatoms chosen from O, N, S and P; it being possible for said alkyl group to be substituted with one or more substituents chosen from OH and halogen atoms (Cl, Br, I and F), and/or to be interrupted with an SiR4R5 group, where R4 and R5, which may be identical or different, represent a C₁-C₆ alkyl group or a phenyl group;

a C₃-C₁₂ cycloalkyl group such as isobornyl or cyclohexane,

a C₃-C₂₀ aryl group such as phenyl,

a C₄-C₃₀ aralkyl group (C₁-C₈ alkyl group) such as 2-phenylethyl; benzyl,

a 4- to 12-membered heterocyclic group containing one or more heteroatoms chosen from O, N and S, the ring being aromatic or nonaromatic,

a heterocycloalkyl group (C₁-C₄ alkyl), such as furfurylmethyl or tetrahydrofurfurylmethyl;

(vi) styrene and derivatives thereof, in particular such as methylstyrene, chlorostyrene or chloromethylstyrene;

(vii) ethylenically unsaturated monomers comprising one or more silicon atoms, such as methacryloxypropyltrimethoxysilane or methacryloxypropyltris(trimethylsiloxy)silane;

(viii) carbon-based or silicone-based macromonomers having at least one polymerizable end group.

This is any polymer, in particular oligomer, comprising, on just one of its ends, an end group, in particular a polymerizable end group, capable of reacting during the polymerization reaction with the monomers under consideration, so as to form the side chains of the polymer; said end group may advantageously be an ethylenically unsaturated group capable of free-radical polymerization with the monomers constituting the backbone. Said macromonomer makes it possible to form the side chains of the copolymer. The polymerizable group of the macromonomer may advantageously be an ethylenically unsaturated group capable of free-radical polymerization. Said polymerizable end group may in particular be a vinyl or (meth)acrylate group. Among the additional macromonomers that may be used, mention may in particular be made of the following, alone or as a mixture, and also the salts thereof:

(a) homopolymers and copolymers of linear or branched C₈-C₂₂ alkyl(meth)acrylate, having a polymerizable end group chosen from vinyl or (meth)acrylate groups, among which mention may be made of macromonomers of poly(2-ethylhexyl acrylate) comprising a mono(meth)acrylate end group; macromonomers of poly(dodecyl acrylate) or of poly(dodecyl methacrylate) comprising a mono(meth)acrylate end group; macromonomers of poly(stearyl acrylate) or of poly(stearyl methacrylate) comprising a mono(meth)acrylate end group. Such macromonomers are in particular described in the patents EP895467 and EP96459;

(b) polyolefins having an ethylenically unsaturated end group, in particular those having a (meth)acrylate end group. As an example of such polyolefins, mention may in particular be made of the following macromonomers, it being understood that they have a (meth)acrylate end group: polyethylene macromonomers, polypropylene macromonomers, polyethylene/polypropylene copolymer macromonomers, polyethylene/polybutylene copolymer macromonomers, polyisobutylene macromonomers; polybutadiene macromonomers; polyisoprene macromonomers; polybutadiene macromonomers; poly(ethylene/butylene)-polyisoprene macromonomers. Such macromonomers are described in particular in U.S. Pat. No. 5,625,005, which mentions ethylene/butylene and ethylene/propylene macromonomers comprising a (meth)acrylate reactive end group. Mention may be made in particular of poly(ethylene/butylene) methacrylate, such as the product sold under the name Kraton Liquid L-1253 by Kraton Polymers;

(c) polydimethylsiloxanes comprising a mono(meth)acrylate end group, and in particular those of formula (IIa) below:

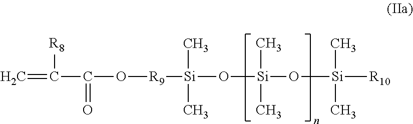

in which:

R₈ denotes a hydrogen atom or a methyl group; preferably methyl;

R₉ denotes a linear or branched, preferably linear, divalent hydrocarbon-based group having from 1 to 10 carbon atoms and optionally containing one or more —O— ether bonds; preferably ethylene, propylene or butylene;

R₁₀ denotes a linear or branched alkyl group having from 1 to 10 carbon atoms, in particular from 2 to 8 carbon atoms; preferably methyl, ethyl, propyl, butyl or pentyl;

n denotes an integer ranging from 1 to 300, preferably ranging from 3 to 200 and preferentially ranging from 5 to 100.

As silicone-based macromonomers, mention may in particular be made of monomethacryloyloxypropylpolydimethylsiloxanes such as those sold under the name PS560-K6 by UCT (United Chemical Technologies Inc.) or under the name MCR-M17 by Gelest Inc.

Among these additional monomers, mention may quite particularly be made of:

C₁-C₁₈ (meth)acrylates, optionally comprising a heteroatom, and which are in particular methyl, ethyl, propyl, isopropyl, n-butyl, isobutyl(methacrylate only), tert-butyl, hexyl, ethylhexyl, octyl, lauryl, isooctyl, isodecyl, dodecyl, cyclohexyl, t-butylcyclohexyl, stearyl, 2-ethylperfluorohexyl, 2-hydroxyethyl, 2-hydroxybutyl, 2-hydroxypropyl, methoxyethyl, ethoxyethyl, methoxypropyl, isobornyl, phenyl, 2-phenylethyl, t-butylbenzyl, benzyl, furfurylmethyl or tetrahydrofurfurylmethyl, methoxypolyoxyethylene (or POE-methyl); POE-behenyl, trifluoroethyl; dimethylaminoethyl, diethylaminoethyl or dimethylaminopropyl(meth)acrylates, and quite particularly methyl, ethyl, n-propyl, isopropyl, n-butyl, t-butyl, cyclohexyl, methoxyethyl, ethoxyethyl, trifluoroethyl, dimethylaminoethyl, diethylaminoethyl, 2-hydroxypropyl or 2-hydroxyethyl(meth)acrylates;

methacrylic acid, (meth)acrylamide, methacryloxypropyltrimethoxysilane, methacryloxypropyltris(trimethylsiloxy)si lane;

and also the salts thereof; and mixtures thereof.

The block polymer according to the invention also comprises a second block, referred to hereinafter as block B.

In one preferred embodiment of the invention, the block polymer is an AB diblock polymer which comprises, in addition to the block A described above, just one additional block, the block B.

In another preferred embodiment of the invention, the block polymer is a triblock polymer which comprises, in addition to the block A described above, an additional block, hereinafter block B, and

(i) either a block comprising both isobutyl acrylate units and acrylic acid units, i.e. a block A′, identical to or different from said block A, generally identical; the block polymer being in the end a triblock polymer of ABA′ structure, generally ABA structure;

(ii) or a block not comprising both isobutyl acrylate units and acrylic acid units, i.e. a block B′, identical to or different from said block B, generally identical; the block polymer being in the end a triblock polymer of BAB′ structure, generally BAB structure.

Preferably, the additional block(s) B, and where appropriate B′, comprise mainly monomers which give the final polymer solubility in the non-aqueous media.

Thus, the block B preferably comprises 70% to 100% by weight, in particular 75% to 90% by weight, or even 80% to 85% by weight, of monomers, alone or as a mixture, chosen from soluble monomers.

The term “soluble monomer” is intended to mean in the present description any monomer of which the homopolymer is in soluble form, i.e. completely dissolved and forming a clear solution, at a concentration of 1% by weight, at ambient temperature (25° C., 1 atm), in isododecane.

The soluble monomers capable of being used may preferably be chosen from the following soluble monomers, alone or as a mixture:

methacrylates of formula CH₂═C(CH₃)—COOR₁ in which R₁ represents a linear or branched C₄-C₂₂ alkyl group, such as isobutyl, tert-butyl, 2-ethylhexyl, lauryl, behenyl or stearyl; or else a cyclic alkyl group having 8 to 30 carbon atoms, such as isobornyl;

acrylates of formula CH₂═CH—COOR₂ in which R₂ represents a linear or branched C₆-C₂₂ alkyl group, such as 2-ethylhexyl, lauryl, behenyl or stearyl; or else a cyclic alkyl group having 8 to 30 carbon atoms, such as isobornyl;

(meth)acrylamides of formula CH₂═C(CH₃)—CONR₃R₄ or CH₂═CH—CONR₃R₄, in which R₃ represents a hydrogen atom or a linear or branched C₁-C₁₂ alkyl group, and R₄ represents a linear or branched C₈-C₁₂ alkyl group, such as an isooctyl, isononyl or undecyl group;

vinyl esters of formula R₅—CO—O—CH═CH₂ in which R₅ represents a linear or branched C₈ to C₂₂ alkyl group;

ethers of vinyl alcohol and of alcohol of formula R₆O—CH═CH₂ in which R₆ represents a linear or branched alkyl group comprising from 8 to 22 carbon atoms;

ethylenic monomers of which the ester group contains silanes or siloxanes, and containing only one silicon atom, such as (meth)acryloxypropyltrimethoxysilane;

carbon-based macromonomers which have a polymerizable end group.

The expression “macromonomer which has a polymerizable end group” is intended to mean any oligomer comprising on just one of its ends a polymerizable end group capable of reacting during the polymerization reaction with ethylenic monomers. The polymerizable group of the macromonomer may advantageously be an ethylenically unsaturated group capable of free-radical polymerization. Said polymerizable end group may in particular be a vinyl or (meth)acrylate (or (meth)acryloxy) group, and preferably a (meth)acrylate group. The term “carbon-based macromonomer” is intended to mean a non-silicone-based macromonomer and in particular an oligomeric macromonomer obtained by polymerization of ethylenically unsaturated non-silicone-based monomer(s), and mainly by polymerization of acrylic and/or non-acrylic vinyl monomers.

As carbon-based macromonomers which have a polymerizable end group, mention may in particular be made of:

(i) homopolymers and copolymers of linear or branched C₆-C₂₂, preferably C_{8-C}₁₈, alkyl(meth)acrylate, having a polymerizable end group chosen from vinyl or (meth)acrylate groups, among which mention may in particular be made of macromonomers of poly(2-ethylhexyl acrylate) comprising a mono(meth)acrylate end group; macromonomers of poly(dodecyl acrylate) or of poly(dodecyl methacrylate) comprising a mono(meth)acrylate end group; macromonomers of poly(stearyl acrylate) or of poly(stearyl methacrylate) comprising a mono(meth)acrylate end group.

Such macromonomers are described in particular in patents EP 895 467 and EP 96459, and in the article by Gillman, Polymer Letters, Vol 5, page 477-481 (1967).

Mention may be made in particular of macromonomers based on poly(2-ethylhexyl acrylate) or poly(dodecyl acrylate) comprising a mono(meth)acrylate end group;

(ii) polyolefins having an ethylenically unsaturated end group, in particular those having a (meth)acrylate end group. As an example of such polyolefins, mention may in particular be made of the following macromonomers, it being understood that they have a (meth)acrylate end group: polyethylene macromonomers, polypropylene macromonomers, polyethylene/polypropylene copolymer macromonomers, polyethylene/polybutylene copolymer macromonomers, polyisobutylene macromonomers; polybutadiene macromonomers; polyisoprene macromonomers; polybutadiene macromonomers; poly(ethylene/butylene)-polyisoprene macromonomers.

Such macromonomers are described in particular in EP 1347013 or else in U.S. Pat. No. 5,625,005, which mentions ethylene/butylene and ethylene/propylene macromonomers comprising a (meth)acrylate reactive end group. Mention may be made in particular of poly(ethylene/butylene) methacrylate, such as the product sold under the name Kraton Liquid L-1253 by Kraton Polymers.

As soluble monomer that is particularly preferred, mention may be made, alone or as a mixture, of:

methacrylates of formula CH₂═C(CH₃)—COOR₁ in which R₁ represents a linear or branched C₄-C₂₂ alkyl group or else a cyclic alkyl group having 8 to 30 carbon atoms;

acrylates of formula CH₂═CH—COOR₂ in which R₂ represents a linear or branched C₆-C₂₂ alkyl group or else a cyclic alkyl group having 8 to 30 carbon atoms.

The soluble monomers that are particularly preferred are 2-ethylhexyl(meth)acrylate, isobornyl(meth)acrylate and isobutyl methacrylate, and mixtures thereof.

The block B may comprise additional monomers, which are therefore insoluble, and which can be present in a proportional from 0 to 30% by weight, in particular from 10% to 25% by weight, or even 15% to 20% by weight, in said block.

These additional monomers are preferably chosen from, alone or as a mixture, the following insoluble monomers and also the salts thereof:

(i) (meth)acrylates of formula CH₂═CHCOOR or CH₂═C(CH₃)COOR in which R represents:

a linear or branched alkyl group comprising 1 to 30 carbon atoms, optionally inserted into which is (are) one or more heteroatoms chosen from O, N, S and P, and/or it being possible for said alkyl group to be optionally substituted with one or more substituents chosen from OH, halogen atoms (Cl, Br, I and F), —NR4R5 groups, where R4 and R5, which may be identical or different, represent hydrogen or a linear or branched C₁-C₆ alkyl group or a phenyl group; and/or polyoxyalkylene, in particular polyoxyethylene and/or polyoxypropylene, groups, said polyoxyalkylene group consisting of 5 to 30 repeating oxyalkylene units;

a C₃-C₁₂cycloalkyl group, it being possible for said cycloalkyl group to comprise in its chain one or more heteroatoms chosen from O, N, S and/or P, and/or to be optionally substituted with one or more substituents chosen from —OH and halogen atoms (Cl, Br, I and F);

a C₄-C₂₀ aryl or C₅-C₃₀ aralkyl group (C₁-C₈ alkyl group);

(ii) (meth)acrylamides of formula CH₂═CHCONR₁R₂ or CH₂═C(CH₃)CONR₁R₂ in which R1 and R2, which may be identical or different, represent H or:

a linear or branched alkyl group comprising from 1 to 18 carbon atoms, optionally inserted into which is (are) one or more heteroatoms chosen from O, N, S and P; it being possible for said alkyl group to be substituted with one or more substituents chosen from OH and halogen atoms (Cl, Br, I and F), and/or to be interrupted with an SiR4R5 group, where R4 and R5, which may be identical or different, represent a C₁-C₆ alkyl group or a phenyl group;

and in particular a methyl, ethyl, propyl, isopropyl, n-butyl, isobutyl, tert-butyl, hexyl, isohexyl, cyclohexyl, ethylhexyl, octyl, isooctyl, decyl, isodecyl, cyclodecyl, dodecyl, cyclododecyl, isononyl, lauryl, t-butylcyclohexyl or stearyl or 2-ethylperfluorohexyl group; or a C₁-C₄ hydroxyalkyl group such as 2-hydroxyethyl, 2-hydroxybutyl or 2-hydroxypropyl; or a (C₁-C₄)alkoxy(C₁-C₄)alkyl group such as methoxyethyl, ethoxyethyl or methoxypropyl,

a C₃-C₁₂ cycloalkyl group, such as isobornyl, or a heterocycloalkyl group (C₁-C₄ alkyl), such as furfurylmethyl or tetrahydrofurfurylmethyl,

a C₄-C₂₀ aryl group such as the phenyl group,

a C₅-C₃₀ aralkyl group (C₁-C₈ alkyl group) such as 2-phenylethyl, t-butylbenzyl or benzyl;

(iii) monomers with ethylenic unsaturation(s) comprising at least one carboxylic, phosphoric or sulfonic acid function, or anhydride function, for instance methacrylic acid, crotonic acid, maleic anhydride, itaconic acid, fumaric acid, maleic acid, styrenesulfonic acid, vinylbenzoic acid, vinylphosphoric acid or acrylamidopropanesulfonic acid, and salts thereof;

(iv) vinyl ethers of formula R₆O—CH═CH₂ or vinyl esters of formula: R₆—COO—CH═CH₂ in which R₆ represents a linear or branched alkyl group comprising from 1 to 22 atoms, or a cyclic alkyl group comprising from 3 to 6 carbon atoms and/or an aromatic group, for example of benzene, anthracene or naphthalene type;

(v) vinyl compounds of formulae CH₂═CH—R9, CH₂═CH—CH₂—R9 or CH₂═C(CH₃)—CH₂—R9 in which R9 is:

a hydroxyl, halogen (Cl or F), NH₂ or acetamide (NHCOCH₃) group;

an OCOR11 group where R11 represents a linear or branched C₂-C₁₂ alkyl group;

an OR10 group where R10 represents a phenyl group or a C₁-C₁₂ alkyl group;

a linear or branched C₁-C₁₈ alkyl group, optionally inserted into which is (are) one or more heteroatoms chosen from O, N, S and P; it being possible for said alkyl group to be substituted with one or more substituents chosen from OH and halogen atoms (Cl, Br, I and F), and/or to be interrupted with an SiR4R5 group, where R4 and R5, which may be identical or different, represent a C₁-C₆ alkyl group or a phenyl group;

a C₃-C₁₂ cycloalkyl group such as isobornyl or cyclohexane,

a C₃-C₂₀ aryl group such as phenyl,

a C₄-C₃₀ aralkyl group (C₁-C₈ alkyl group) such as 2-phenylethyl; benzyl,

a 4- to 12-membered heterocyclic group containing one or more heteroatoms chosen from O, N and S, the ring being aromatic or nonaromatic,

a heterocycloalkyl group (C₁-C₄ alkyl), such as furfurylmethyl or tetrahydrofurfuryl methyl;

(vi) styrene and derivatives thereof, in particular such as methylstyrene, chlorostyrene or chloromethylstyrene;

(vii) ethylenically unsaturated monomers comprising at least two silicon atoms, such as methacryloxypropyltris(trimethylsiloxy)silane.

Among these additional monomers, mention may quite particularly be made of isobutyl acrylate, methyl(meth)acrylate, methacrylic acid and methacryloxypropyltris(trimethylsiloxy)silane.

The block, or blocks, comprising both isobutyl acrylate and acrylic acid (block A and optionally A′) represent(s) in total preferably 5% to 55% by weight, in particular 10% to 50% by weight, or even 15% to 47% by weight, even better still 20% to 44% by weight, of the total weight of the block polymer.

The additional block, or additional blocks (block B and optionally B′) represent(s) in total preferably 45% to 95% by weight, in particular 50% to 90% by weight, or even 53% to 85% by weight, even better still 56% to 80% by weight, of the total weight of the block polymer.

Preferably, the polymer according to the invention has a modulus E′ measured by DMTA of greater than or equal to 550 MPa. The equipment used is a DMA-2980 from TA Instruments; the frequency is 1 Hz, the scan rate is 3° C. per minute, between −100° C. and +200° C.

The polymer according to the invention preferably has two glass transition temperatures.

Preferably, the block A comprising the isobutyl acrylate and the acrylic acid, or the set of these blocks if there are more than one, has a number-average molecular weight (Mn) of between 5000 and 50 000 g/mol, preferably between 7000 and 45 000.

Preferably, the block B not comprising the isobutyl acrylate+acrylic acid mixture, or the set of these blocks if there are more than one, has a number-average molecular weight (Mn) of between 50 000 and 150 000 g/mol, preferably between 55 000 and 120 000, or even between 60 000 and 100 000.

Preferably, the block polymer according to the invention has a polydispersity index (Ip=Mw/Mn) of between 1.01 and 2.2.

The weight-average molar mass (Mw) and number-average molar mass (Mn) are determined by gel permeation liquid chromatography (THF solvent, calibration curve established with linear polystyrene standards, refractometric detector).

The polymers according to the invention can be prepared according to the methods known to those skilled in the art. A general preparation process is described before the examples.

The block polymer according to the invention is preferentially soluble in a non-aqueous medium.

The term “soluble” is intended to mean that the polymer does not form a precipitate, but a clear solution, in said non-aqueous medium, at 25° C., 1 atm, at a concentration of 1% by weight, in said non-aqueous medium; preferably at a concentration of 5% by weight, or even of 10% by weight.

Said non-aqueous medium comprises, preferably exclusively, one or more non-aqueous compounds that are liquid at 25° C., having an overall solubility parameter according to the Hansen solubility space of less than or equal to 20 (MPa)^{1/2}, or a mixture of such compounds.

The overall solubility parameter δ according to the Hansen solubility space is defined in the article “Solubility parameter values” by Grulke in the book “Polymer Handbook”, 3rd Edition, Chapter VII, pages 519-559, by the relationship:

δ=(d_{D}²+dₚ²+d_{H}²)^{1/2}

in which:

d_{D} characterizes the London dispersion forces arising from the formation of dipoles induced during molecular impacts,

d_{P} characterizes the Debye interaction forces between permanent dipoles, and

d_{H} characterizes the forces of specific interactions (such as hydrogen bonding, acid/base, donor/acceptor, etc.).

The definition of solvents in the Hansen three-dimensional solubility space is described in the article by Hansen: “The three dimensional solubility parameters”, J. Paint Technol. 39, 105 (1967).

Among the non-aqueous liquid compounds having an overall solubility parameter according to the Hansen solubility space of less than or equal to 20 (MPa)^{1/2}, mention may be made of liquid fatty substances, in particular oils, which may be chosen from natural or synthetic, carbon-based, or even hydrocarbon-based, optionally fluoro, optionally branched oils, alone or as a mixture. Mention may in particular be made, alone or as a mixture, of:

vegetable oils made up of fatty acid esters of polyols, in particular triglycerides, such as sunflower oil, sesame oil, rapeseed oil, macadamia oil, soy oil, sweet almond oil, beauty-leaf oil, palm oil, grapeseed oil, maize oil, arara oil, cottonseed oil, apricot oil, avocado oil, jojoba oil, olive oil or cereal germ oil;

linear, branched or cyclic esters having 5 to 30 carbon atoms; and more particularly esters of formula RCOOR′ in which R represents the residue of a carboxylic acid comprising 2 to 19 carbon atoms and R′ represents a hydrocarbon-based chain comprising from 3 to 20 carbon atoms, such as acetates, palmitates, adipates, myristates and benzoates, in particular diisopropyl adipate and isopropyl myristate;

hydrocarbons and in particular volatile or non-volatile, linear, branched and/or cyclic C₅-C₆₀ alkanes, for instance C₅-C₆₀ isoparaffins, which are optionally volatile, such as isododecane, parleam (hydrogenated polyisobutene), isohexadecane, cyclohexane or isopars; or else liquid paraffins, liquid petroleum jelly, or hydrogenated polyisobutylene;

ethers having 6 to 30 carbon atoms;

ketones having 6 to 30 carbon atoms;

aliphatic monoalcohols having 6 to 30 carbon atoms, the hydrocarbon-based chain not comprising a substitution group, such as oleyl alcohol, decanol, dodecanol and octadecanol, octyldodecanol and linoleyl alcohol;

polyols, in particular having 6 to 30 carbon atoms, such as hexylene glycol.

Preferably, the non-aqueous medium comprises, alone or as a mixture, isododecane, parleam, butyl acetate, isononyl isononanoate and/or octyldodecanol.

The block polymers according to the invention can be used in a cosmetic composition which comprises, moreover, a cosmetically acceptable medium. They may be present, alone or as a mixture, in the cosmetic compositions according to the invention in an amount of from 0.1% to 50% by weight, preferably 0.5% to 40% by weight, in particular 1% to 30% by weight, of dry matter relative to the total weight of the composition.

The composition may thus comprise, depending on the intended application, constituents that are common for this type of composition.

Preferably, said composition comprises at least one non-aqueous medium as defined above.

It may also comprise other constituents in particular chosen from waxes, oils, gums and/or pasty fatty substances, of vegetable, animal, mineral or synthetic origin, or even which are silicone-based, and mixtures thereof.

Among the waxes that may be present in the composition according to the invention, mention may be made, alone or as a mixture, of hydrocarbon-based waxes such as beeswax; carnauba wax, candelilla wax, ouricury wax, Japan wax, cork fibre wax or sugar cane wax; paraffin wax, lignite wax; microcrystalline waxes; lanolin wax; Montan wax; ozokerites; polyethylene waxes; waxes obtained by Fischer-Tropsch synthesis; hydrogenated oils, fatty esters and glycerides that are solid at 25° C. It is also possible to use silicone waxes, among which mention may be made of alkyl or alkoxy polymethylsiloxanes and/or polymethylsiloxane esters.

The composition according to the invention may also comprise carbon-based, hydrocarbon-based, fluoro and/or silicone oils of mineral, vegetable or synthetic origin, alone or as a mixture, provided that they form a homogeneous and stable mixture and that they are compatible with the intended use. Among the oils that may be present in the composition according to the invention, mention may be made, alone or as a mixture, of hydrocarbon-based oils such as liquid paraffin or liquid petroleum jelly; perhydrosqualene; arara oil; sweet almond oil, beauty-leaf oil, palm oil, castor oil, avocado oil, jojoba oil, olive oil or cereal germ oil; lanolic acid, oleic acid, lauric acid or stearic acid esters; alcohols such as oleyl alcohol, linoleyl alcohol, linolenyl alcohol, isostearyl alcohol or octyldodecanol. Silicone oils may also be mentioned, such as optionally phenylated PDMSs, for instance phenyl trimethicones. Use may also be made of volatile oils, such as cyclotetradimethylsiloxane, cyclopentadimethylsiloxane, cyclohexadimethylsiloxane, methylhexyldimethylsiloxane, hexamethyldisiloxane or isoparaffins.

The composition according to the invention may also comprise one or more colorants chosen from pulverulent compounds and/or dyes which are liposoluble or water-soluble.

The pulverulent compounds may be chosen from the pigments and/or nacres and/or fillers normally used in cosmetic or pharmaceutical compositions. Advantageously, the pulverulent compounds represent 0.1% to 50% of the total weight of the composition and better still from 1% to 40%.

The pigments may be white or coloured, inorganic and/or organic, and interference or non-interference pigments. Among the inorganic pigments that may be mentioned are titanium dioxide, optionally surface-treated, zirconium oxides or cerium oxides, and also iron oxides or chromium oxides, manganese violet, ultramarine blue, chromium hydrate and ferric blue. Among the organic pigments that may be mentioned are carbon black, pigments of D & C type and lakes based on cochineal carmine or on barium, strontium, calcium or aluminium.

The nacreous pigments may be chosen from white nacreous pigments such as mica coated with titanium or with bismuth oxychloride, coloured nacreous pigments such as titanium mica with iron oxides, titanium mica in particular with ferric blue or with chromium oxide, titanium mica with an organic pigment of the abovementioned type, and also nacreous pigments based on bismuth oxychloride. The fillers may be inorganic or organic, and lamellar or spherical. Mention may be made of talc, mica, silica, kaolin, powders of Nylon and of polyethylene, of poly-β-alanine and of polyethylene, Teflon, lauroyllysine, starch, boron nitride, powders of tetrafluoroethylene polymers, hollow microspheres such as Expancel (Nobel Industrie), Polytrap (Dow Corning) and silicone resin microbeads (for example Tospearls from Toshiba), precipitated calcium carbonate, magnesium carbonate, magnesium hydrogen carbonate, hydroxyapatite, hollow silica microspheres (Silica Beads from Maprecos), glass or ceramic microcapsules, and metal soaps derived from organic carboxylic acids having from 8 to 22 carbon atoms and preferably from 12 to 18 carbon atoms, for example zinc stearate, magnesium stearate or lithium stearate, zinc laurate or magnesium myristate.

The liposoluble dyes are, for example, Sudan red, DC Red 17, DC Green 6, β-carotene, soybean oil, Sudan brown, DC Yellow 11, DC Violet 2, DC Orange 5 and quinoline yellow. They may represent 0.01% to 20% of the weight of the composition and better still from 0.1% to 6% by weight. The water-soluble dyes are, for example, beetroot juice or methylene blue, and may represent up to 6% of the total weight of the composition.

The composition may also comprise any additive normally used in the cosmetics field, such as antioxidants, fragrances, essential oils, preservatives, cosmetic active agents, moisturizers, vitamins, essential fatty acids, ceramides, sunscreens, polymers, thickeners, gelling agents or surfactants. Needless to say, those skilled in the art will take care to select this or these additive(s), and/or the amount thereof, such that the advantageous properties of the composition according to the invention are not, or are not substantially, adversely affected by the envisaged addition.

The composition according to the invention may be in the form of a suspension or a dispersion, in particular of oil-in-water by means of vesicles; an optionally thickened or even gelled oily solution; an oil-in-water, water-in-oil or multiple emulsion; a gel or a mousse; an oily or emulsified gel; a dispersion of vesicles, in particular lipid vesicles; a two-phase or multiphase lotion; a spray; a lotion, a cream, a salve, a soft paste, an ointment, a solid that has been cast or moulded in particular as a stick or in a dish, or a compacted solid.

Those skilled in the art may select the appropriate galenical form, and also the method for preparing it, on the basis of their general knowledge, taking into account both the nature of the constituents used, in particular their solubility in the support, and the intended use of the composition.

The cosmetic composition according to the invention may be in the form of a product for caring for and/or making up bodily or facial skin, the lips, the eyelashes or the nails; of an anti-sun or self-tanning product, or of a hair product, in particular for caring for, making up, retaining the form of, shaping, cleansing, conditioning, dyeing or straightening the hair.

The composition according to the invention has a particularly advantageous application in the make-up field, in particular the field of making up the lips, the eyelashes, the nails and/or the face. It may therefore advantageously be in the form of a make-up composition, in particular a mascara, eyeliner, lipstick, face powder, eyeshadow, foundation or nail varnish composition, of a facial care composition or of an anti-sun composition.

A subject of the invention is also a cosmetic process for treating keratin materials, in particular bodily or facial skin, the lips, the eyelashes, the nails and/or the hair, comprising the application to said materials of a cosmetic composition as defined previously.

This process makes it possible in particular to make up the skin, the lips, the eyelashes, the nails and/or the hair, preferentially the skin, the lips, the nails and/or the eyelashes.

The invention is illustrated in more detail in the following examples.

The polymerization process can be described in the following general way:

The mixture of monomers, initiator and RAFT agent is placed in solution in a solvent, and then transferred into a 250 ml tube. The solution is degassed by means of 3 cycles of freezing/thawing under vacuum (vacuum of 0.005 mmHg). The tube is sealed and immersed in a thermostated bath for the duration indicated. After the polymerization, solvent is again added in order to dissolve the polymer, and the polymer solution is precipitated from a non-solvent in order to recover the polymer.

Synthesis of RAFT Agents

Literature: Australian journal of chemistry, 2009, vol 62, No.11, page 391, scheme 11 and page 1453, scheme 14.

### a) RAFT agent ST902-33: 4-cyano-4-(dodecylsulfanylthiocarbonyl)sulfanylpentanoic acid (Polymer 2005, 46, 8458-8468)

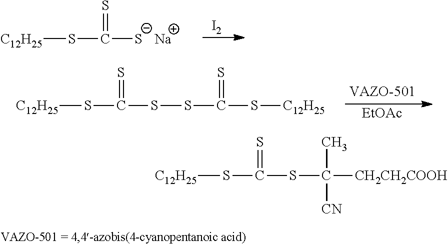

n-Dodecylthiol (15.4 g, 76 mmol) is mixed with a suspension of sodium hydride (60% in oil) (3.15 g, 79 mmol) in 150 ml of diethyl ether, at 5-10° C. The mixture is cooled to 0° C. and carbon disulfide (6.0 g, 79 mmol) is added in order to obtain a precipitate of sodium S-dodecyl trithiocarbonate, which is recovered by filtration and used in the following step, without purification.

Solid iodine (6.3 g, 0.025 mol) is added little by little to a suspension of sodium S-dodecyl trithiocarbonate (14.6 g, 0.049 mol) in 100 ml of diethyl ether. Mixing is carried out at 25° C. for 1 hour, and the yellow sodium iodide formed is removed by filtration.

The yellow-brown filtrate is washed with an aqueous solution of sodium thiosulfate in order to remove the excess iodine, then dried over sodium sulfate, and evaporated so as to obtain bis(dodecylsulfanylthiocarbonyl) disulfide (13.6 g),

Melting point: 33-35° C.

¹H NMR (CDCl₃): δ (ppm) 0.89, t, 6H; 1.30, br s, 36H; 1.71, m, 4H; 3.29, t, 4H.

A solution of 4,4′-azobis(4-cyanopentanoic acid) (2.10 g, 0.0075 mol) and of bis-(dodecylsulfanylthiocarbonyl)disulfide (2.77 g, 0.005 mol) in 50 ml of ethyl acetate is refluxed for 18 hours. The volatile compounds are removed under vacuum, and the crude product is then extracted with water in order to obtain 4-cyano-4-(dodecylsulfanylthiocarbonyl)sulfanylpentanoic acid, in the form of a pale yellow

solid (3.65 g, yield: 87%).

Melting point: 58-59° C., after recrystallization with hexane.

¹H NMR (CDCl₃): δ (ppm) 0.89 (t, 3H, CH₃); 1.28 (br s, 18H); 1.72 (m, 2H); 1.89 (s, 3H, CH₃); 2.40-2.80 (m, 4H, CH₂CH₂); 3.38 (t, 2H, CH₂S).

### b) RAFT agent: ST902-57A

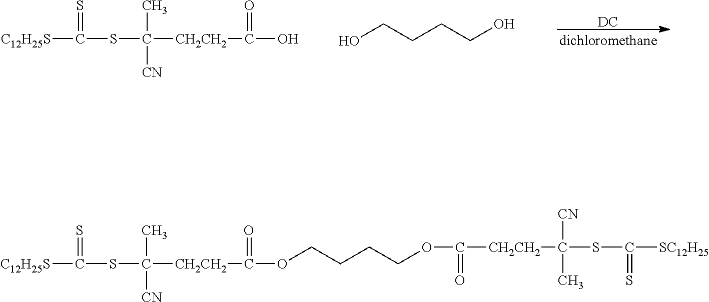

4-Cyano-4-(dodecylsulfanylthiocarbonyl)sulfanylpentanoic acid (8.1 g, 20.1 mmol), 1,4-butanediol (0.9 g, 10 mmol) and dicyclohexylcarbodiimide (4.95 g, 24.0 mmol) are mixed in 60 ml of dichloromethane, in the presence of DMAP (N,N-dimethylaminopyridine, catalytic amount), for 1 hour at 25° C. The solvent is removed and the crude product is passed over a silica chromatography column, with a 2/5 ethyl acetate/n-hexane mixture as eluent; 6.2 g of desired product are obtained (yield 72%), in the form of a yellow oil which solidifies in the cold. ¹H NMR (CDCl₃) : complies.

### c) RAFT agent: GXL090612

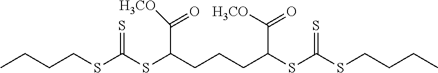 [SMILES: CCCCSC(=S)SC(CCCC(SC(=S)SCCCC)C(=O)OC)C(=O)OC]

Known compound: A. M. Bivigou-Koumba, J. Kristen, A. Laschewsky, P. Müller-Buschbaum, C. M. Papadakis. Macromol. Chem. Phys. 2009, 210, 565-578.

### EXAMPLE 1

A diblock polymer with a main block (35/35 isobornyl acrylate/isobornyl methacrylate) and a secondary block (25/5 isobutyl acrylate/acrylic acid) is prepared.

Main block: theoretical Mn: 70 000 g/mol

Secondary block: theoretical Mn: 30 000 g/mol

The RAFT agent used is ST902-33 of formula:

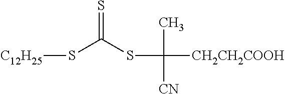

RAFT agent (I)—ST902-33; C₁₉H₃₃NO₂S₃: MW 403

The initiator used is AIBN (azobisisobutyronitrile).

Block 1:

| | | |
|---|---|---|
| Isobornyl methacrylate | 35.01 | g |
| Isobornyl acrylate | 35.01 | g |
| AIBN | 101.8 | mg |
| RAFT agent | 208 | mg |
| Solvent | 30 | ml |

Reaction conditions: 6 hours at 55° C.

The solvent is ethyl acetate (30 ml).

The non-solvent is chloroform (200 ml).

A first block is obtained (57 g), yield 81.2%, with Mn=71 200 g/mol and Ip=1.3.

Diblock Polymer:

| | | |
|---|---|---|
| block 1 | 25.0 | g |
| isobutyl acrylate | 16.1 | g |
| acrylic acid | 3.2 | g |
| AIBN | 33.1 | mg |

| | |
|---|---|
| Solvent | 75 ml + 250 ml |

Reaction conditions: 16 hours at 60° C.

The solvent is benzene (75 ml) subsequently diluted with THF (250 ml).

The non-solvent is acetonitrile (2.5 litres).

The desired diblock copolymer is obtained (38.2 g), yield 86.5%, with Mn=110 200 g/mol and Ip=1.5.

E′ modulus 1 Hz=816 MPa

Tg=62° C. and 180° C.

### EXAMPLE 2

A triblock polymer with a central block (35/35 isobornyl acrylate/isobornyl methacrylate) and two identical external blocks (12.5/2.5 isobutyl acrylate/acrylic acid for each block) is prepared.

Central block: theoretical Mn: 70 000 g/mol

External blocks: theoretical Mn: 15 000 g/mol (×2)

The RAFT agent used is ST902-57A

The initiator used is AIBN (azobisisobutyronitrile).

Central Block:

| | | |
|---|---|---|
| Isobornyl methacrylate | 35.01 | g |
| Isobornyl acrylate | 35.03 | g |
| AIBN | 100 | mg |
| RAFT agent | 430 | mg |
| Solvent | 30 | ml |

Reaction conditions: 5 hours and 45 minutes at 55° C.

The solvent is ethyl acetate (30 ml).

The non-solvent is chloroform (250 ml) and methanol (3 litres).

A first block is obtained (54 g), yield: 76.6%, with Mn=71 300 g/mol and Ip=1.2.

Triblock Polymer:

| | | |
|---|---|---|
| central block | 35 | g |
| isobutyl acrylate | 13.2 | g |
| acrylic acid | 2.7 | g |
| AIBN | 30 | mg |

| | |
|---|---|
| Solvent | 120 ml + 120 ml |

Reaction conditions: 22 hours at 60° C.

The solvent is chloroform (120 ml)+THF (120 ml).

The non-solvent is acetonitrile (2.5 litres).

The desired triblock copolymer is obtained (44.1 g), yield 86.6%, with Mn=96 400 g/mol and Ip=1.2.

E′ modulus 1 Hz=720 MPa

Tg=48° C. and 136° C.

### EXAMPLE 3

### Comparative

A diblock polymer with a main block (29/29 isobornyl acrylate/isobornyl methacrylate) and a secondary block (42 isobutyl acrylate) is prepared.

The synthesis is carried out in two steps: synthesis of the poly(isobutyl acrylate) block and then of the second block of poly(isobornyl acrylate-co-isobornyl methacrylate).

### 1st step: synthesis of poly(isobutyl acrylate) (Mn=42 000)

150 g of isobutyl acrylate are placed in a round-bottomed flask, followed by 220 μl of initiator (ethyl 2-bromoisobutyrate) and 313 μl of ligand (PMDETA or pentamethyldiethylenetriamine). The catalyst (215 mg of CuBr(I)) is introduced and the mixture is left under an argon stream in an oil bath at 90° C., for 6 hours. The solution is then poured into 4 litres of a 50/50 water/ethanol mixture, from which the polymer precipitates. The water/EtOH solution is removed, and the polymer is recovered and then redissolved in THF. The solution is filtered over neutral alumina, and the solvent is then evaporated off under reduced pressure.

### 2nd step: synthesis of poly(isobutyl acrylate)-b-poly(isobornyl acrylate-co-isobornyl methacrylate)

9.6 g of previously prepared polymer are placed in a round-bottomed flask under argon, and 32.5 ml of butyl acetate are added. The mixture is left to stir until complete dissolution is obtained. 20 g of isobornyl methacrylate, 20 g of isobornyl acrylate and then 0.048 ml of ligand are added. The catalyst (33 mg of CuBr(I)) is introduced and the mixture is left under an argon stream in an oil bath at 90° C., for 52 hours. The solution is then poured into 4 litres of a 50/50 water/ethanol mixture, from which the polymer precipitates. The water/EtOH solution is removed, and the polymer is recovered and then redissolved in THF. The solution is filtered over neutral alumina, followed by filtration on paper, and the polymer obtained is dried. The desired polymer is obtained in the form of a light yellow powder. Mw=100 000, Ip=1.3.

### EXAMPLE 4

### Evaluation

The resistance to olive oil is measured with a drop of olive oil placed on a dry polymer film.

A polymer film is prepared from 0.5 ml of a solution at 20% by weight in a solvent, spread on a 2.5×7.5 cm glass plate and dried at ambient temperature (25° C.) for 24 hours. Next, 1 ml of olive oil is spread on the polymer film. After 3 hours, the excess oil is wiped from the film and the tack is determined by touch.

Examples 1 and 2: the solvent is butyl acetate

Comparative example (Example 3): the solvent is isododecane

It is noted that the film formed with the polymer of the comparative example (Example 3) is tacky after 3 hours, whereas the films formed with the polymers of Examples 1 or 2 are non-tacky after 3 hours.

The tack reflects the sensitivity of the polymer to olive oil. The higher this sensitivity, the more easily the deposit will be modified during meals, bringing about a worse wear property. The wear property of the polymers according to the invention is better than that of the comparative polymer.

### EXAMPLE 5

### Cosmetic Composition

25 g of the polymer of Example 1 (obtained in dry form) are dissolved in 70 g of a butyl acetate/ethyl acetate mixture (70/30 by weight).

The solution obtained is then applied to the nails. The film exhibits good wear over time.

### EXAMPLE 6

### Cosmetic Composition

25 g of the polymer of Example 2 (obtained in dry form) are dissolved in 70 g of a butyl acetate/ethyl acetate mixture (70/30 by weight).

The solution obtained is then applied to the nails. The film exhibits good wear over time.

### EXAMPLE 7

### Cosmetic Composition

A lip gloss is prepared, having the following composition (% by weight):

| | |
|---|---|
| polybutene | 34% |
| isononyl isononanoate | 4% |
| octyldodecanol | 10% |
| silica (Aerosil R972) | 5% |
| polymer of Example 2 | 14% DM |
| isododecane | 20% |
| tridecyl trimellitate | qs 100% |

The mixture obtained is then applied to the lips. The deposit exhibits good cosmetic properties.

1. Diblock or triblock polymer of AB, ABA or BAB structure, comprising at least one block A which comprises isobutyl acrylate and acrylic acid.

2. Polymer according to claim 1, in the form of an ethylenic polymer.

3. Polymer according to claim 1, wherein, in the block A, the isobutyl acrylate represents 75% to 99.5% by weight of the total weight of the isobutyl acrylate+acrylic acid mixture; and the acrylic acid represents 0.5% to 25% by weight of the total weight of the isobutyl acrylate+acrylic acid mixture.

4. Polymer according to claim 1, wherein the isobutyl acrylate+acrylic acid mixture represents 50% to 100% by weight of the total weight of said block A.

5. Polymer according to claim 1, wherein said block A comprises at least one additional monomer chosen from, alone or as a mixture:
C₁-C₁₈ (meth)acrylates, optionally comprising a heteroatom;
methacrylic acid, (meth)acrylamide, methacryloxypropyltrimethoxysilane, methacryloxypropyltris(trimethylsiloxy)silane;
and the salts thereof.

6. Polymer according to claim 1, also comprising a second block, or block B, which comprises 70% to 100% by weight of monomers, alone or as a mixture, chosen from the following soluble monomers, alone or as a mixture:
methacrylates of formula CH₂═C(CH₃)—COOR₁ in which R₁ represents a linear or branched C₄-C₂₂ alkyl group or else a cyclic alkyl group having 8 to 30 carbon atoms;
acrylates of formula CH₂═CH—COOR₂ in which R₂ represents a linear or branched C_{6-C}₂₂ alkyl group or else a cyclic alkyl group having 8 to 30 carbon atoms;
(meth)acrylamides of formula CH₂═C(CH₃)—CONR₃R₄ or CH₂═CH—CONR₃R₄, in which R₃ represents a hydrogen atom or a linear or branched C₁-C₁₂ alkyl group, and R₄ represents a linear or branched C₈-C₁₂ alkyl group;
vinyl esters of formula R₅—CO—O—CH═CH₂ in which R₅ represents a linear or branched C₈ to C₂₂ alkyl group;
ethers of vinyl alcohol and of alcohol of formula R₆O—CH═CH₂ in which R₆ represents a linear or branched alkyl group comprising from 8 to 22 carbon atoms;
ethylenic monomers of which the ester group contains silanes or siloxanes, and containing only one silicon atom;
carbon-based macromonomers which have a polymerizable end group.

7. Polymer according to claim 6, in which the soluble monomers are chosen from the following monomers, alone or as a mixture:
methacrylates of formula CH₂═C(CH₃)—COOR₁ in which R₁ represents a linear or branched C₄-C₂₂ alkyl group or else a cyclic alkyl group having 8 to 30 carbon atoms;
acrylates of formula CH₂═CH—COOR₂ in which R₂ represents a linear or branched C₆-C₂₂ alkyl group or else a cyclic alkyl group having 8 to 30 carbon atoms.

8. Polymer according to claim 1, wherein the block, or blocks, comprising both isobutyl acrylate and acrylic acid represent(s) in total 5% to 55% by weight of the total weight of the block polymer.

9. Polymer according to claim 1, wherein the block, or blocks, comprising isobutyl acrylate and acrylic acid has (have) a number-average molecular weight (Mn) of between 5000 and 50 000 g/mol.

10. Polymer according to claim 1, wherein has a polydispersity index (Ip=Mw/Mn) of between 1.01 and 2.2.

11. Cosmetic composition comprising, in a cosmetically acceptable medium, at least one polymer according to claim 1.

12. Composition according to claim 11, in which the polymer is present, alone or as a mixture, in an amount of from 0.1% to 50% by weight of dry matter relative to the total weight of the composition.

13. Composition according to claim 11, wherein the cosmetically acceptable medium comprises at least one ingredient chosen from waxes, oils, gums, pasty fatty substances, of vegetable, animal, mineral or synthetic origin, or even which are silicone-based; colorants chosen from pulverulent compounds and/or dyes which are liposoluble or water-soluble; antioxidants, fragrances, essential oils, preservatives, cosmetic active agents, moisturizers, vitamins, essential fatty acids, ceramides, sunscreens, polymers, thickeners, gelling agents and surfactants.

14. Composition according to claim 11, in the form of a product for caring for and/or making up bodily or facial skin, the lips, the eyelashes or the nails; of an anti-sun or self-tanning product, or of a hair product.

15. Cosmetic process for treating keratin materials, comprising the application to said materials of a cosmetic composition as defined in claim 11.

16. Polymer according to claim 1, in the form of a linear ethylenic polymer.

17. Polymer according to claim 2, wherein, in the block A, the isobutyl acrylate represents 75% to 99.5% by weight of the total weight of the isobutyl acrylate+acrylic acid mixture; and the acrylic acid represents 0.5% to 25% by weight of the total weight of the isobutyl acrylate+acrylic acid mixture.

18. Polymer according to claim 1, wherein, in the block A, the isobutyl acrylate represents 78% to 95% by weight of the total weight of the isobutyl acrylate+acrylic acid mixture; and the acrylic acid represents 5% to 22% by weight of the total weight of the isobutyl acrylate+acrylic acid mixture.

19. Polymer according to claim 2, wherein the isobutyl acrylate+acrylic acid mixture represents 50% to 100% by weight of the total weight of said block A.

20. Polymer according to claim 3, wherein the isobutyl acrylate+acrylic acid mixture represents 50% to 100% by weight of the total weight of said block A.
